(19) United States
(12) Patent Application Publication
(10) Pub. No.: US 2008/0228213 A1
(43) Pub. Date: Sep. 18, 2008

(54) Variable size trocar

(75) Inventors: Derek C. Blakeney, Saline, MI (US); Randal J. Kadykowski, South Lyon, MI (US); Seiji Maeda, Tokyo (JP); Ken Yamatani, Tokyo (JP)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignee: Terumo Cardiovascular Systems Corporation and Olympus Medical Systems Corporation

(21) Appl. No.: 11/724,616

(22) Filed: Mar. 15, 2007

(51) Int. Cl. A61B 17/34

(52) U.S. Cl. 606/185

(57) ABSTRACT

A trocar includes a guide tube axially movable within a securing member. The securing member has an annular member having an inside circumference which is complementary with the outside circumference of the guide tube so that the guide tube is axially movable within the annular member.

### CROSS-REFERENCE TO RELATED APPLICATIONS AND STATEMENT REGARDING SPONSORED RESEARCH

Not Applicable.

### BACKGROUND OF THE INVENTION

Various devices and methods for drawing and removing the subcutaneous vessel by using an endoscope and an apparatus thereof are known.

A trocar is a surgical instrument placed within an opening in a patient to permit the insertion of a surgical device such as an endoscope, forceps, or electric cautery into the opening in the patient. Patients vary in size, weight and physique. As such, the thickness of the superficial fat layer and the tightness of tissue and musculature are among the most challenging variables that are faced when a trocar is to be inserted into the patient.

The present invention provides a trocar that meets these needs.

### SUMMARY OF THE INVENTION

In one aspect, there is provided a trocar used for introducing an endoscope, forceps, electric cautery or other device into a body cavity in surgery.

A variable size trocar includes a securing member that has an annular opening and a guide tube that is slidingly or longitudinally movable in the annular opening in the securing member. The guide tube has a desired outer dimension that is complementary with the inner dimension of the annular member. At least a portion of the guide tube is longitudinally movable with respect to the annular member. The securing member can accept guide tubes that have one or more different lengths and/or different diameters.

In a particular aspect, the trocar can include first and second guide tubes where the second guide tube can have a slight inner diameter tapered surface such that the first guide tube can be held by the taper of the second guide tube.

In another particular aspect, the first guide tube can include a detent that is matingly received in a detent in the second guide tube.

Also, in another particular aspect, the trocar can include a lockable guide tube. The lockable guide tube can have one or more grooves or detents on its outer surface. The trocar can include a protrusion that is at least partially engaged by at least one detent.

In yet another aspect, the trocar can include a first guide tube and a second guide tube that is co-axially positioned within the first guide tube, where least one of the first and second guide tubes can be circumferentially rotated relative to one another.

In a particular embodiment, the first guide tube can include one or more projections and the second guide tube is configured to receive the one or more projections. The second guide tube can include one or more apertures that receive the projections.

In one particular embodiment, the aperture in the second guide tube has a generally spirally extending configuration. Also, in certain embodiments, the spirally extending aperture can have one or more locking grooves that extend at an angle from the spirally extending aperture on the outer surface of the second guide tube. The projections are at least temporarily secured at least within the grooves and prevent the second guide tube from longitudinally moving with respect to the first guide tube.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a structure diagram, partially in phantom, showing a side elevation view of one embodiment of a variable size trocar.

FIG. 2 is a structure diagram, partially in phantom, showing a side elevation view of one embodiment of a variable size trocar having telescoping guide tubes.

FIG. 3 is a structure diagram, partially in phantom, showing a side elevation view of one embodiment of a variable size trocar having score lines.

FIG. 4 is a structure diagram, partially in phantom, showing a side elevation view of one embodiment of a variable size trocar having telescoping guide tubes and mating detents.

FIG. 5A is a structure diagram, partially in phantom, showing a side elevation view of another embodiment of a variable size trocar in a closed and locked position.

FIG. 5B is a structure diagram, partially in phantom, showing a side elevation view of the embodiment shown in FIG. 5A in an open and unlocked position.

FIG. 6A is a structure diagram, partially in phantom, showing an exploded perspective view of another embodiment of a variable size trocar.

FIG. 6B is a partial cross-sectional view of the embodiment shown in FIG. 6A in an assembled, closed and locked position.

FIG. 7A is a structure diagram showing a perspective view of yet another embodiment of a variable size trocar having interlocking inner and outer guide tubes.

FIG. 7B is a structure diagram showing a perspective view of the inner guide tube shown in FIG. 7A.

### DETAILED DESCRIPTION OF THE PREFERRED EMBODIMENT(S)

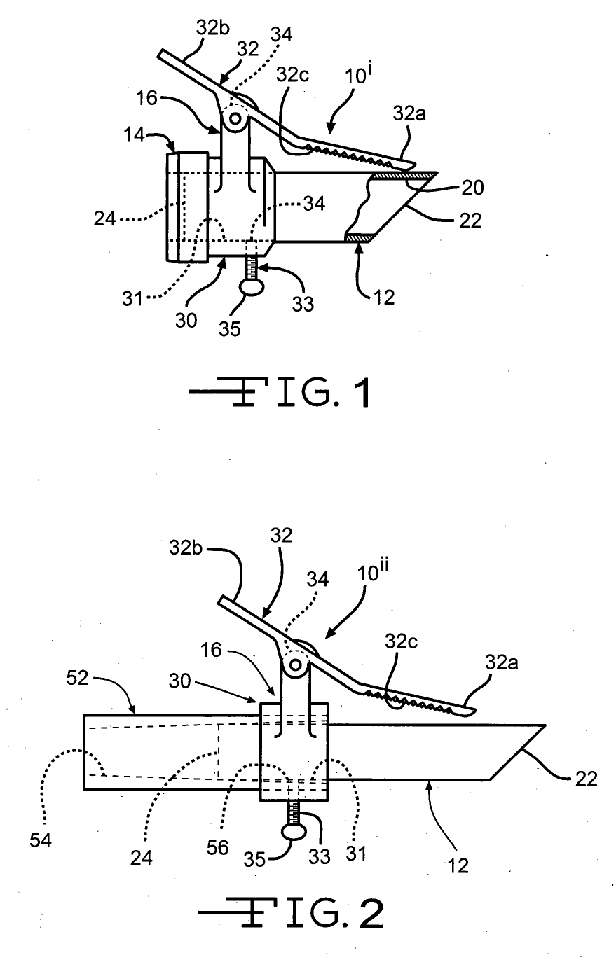

FIG. 1 is a side elevation showing one embodiment of a trocar 10ⁱ. The trocar 10ⁱ includes a guide tube 12 and a securing member 16.

The guide tube 12 defines an opening 20 for receiving a surgical tool or instrument. In the embodiment shown, for ease of illustration, the guide tube 12 will be generally illustrated as having a circular cross-sectional shape, but it should be understood that the guide tube can have other suitable shapes such as, for example, elliptical, oval, or any shape that will accommodate the clinician's instruments.

The guide tube 12 has a distal end 22 and a proximal end 24. In the embodiment shown, the distal end 22 has an angled shape to facilitate insertion of the distal end 22 into an open wound in the patient. While the embodiment shown illustrates the distal end 22 as being at an angle of about 45° with respect to a longitudinal axis defined by the guide tube 12, the angle can be at an angle other than 45°.

There is no special limitation on the material for the guide tube 12 and/or securing member 16 except that the material has a strength that is able to withstand the forces applied by the securing member 16 against the guide tube 12, as further described below. In non-limiting examples, suitable materials include: metallic materials such as stainless steel, an aluminum alloy, a super high-resilience metal and a shape-memory alloy; and, synthetic resins such as polyamide, poly(vinyl chloride), polycarbonate, ABS (acrylonitrile butadiene styrene copolymer), polyethylene, polypropylene, fluorocarbon resin and acrylic resin. The above-mentioned synthetic resins which are not electrically conductive are useful, and polycarbonate and ABS, in certain embodiments, are especially useful, taking into account the use of electric devices such as an electric cautery.

The securing member 16 can accept guide tubes 12 that have different lengths and/or diameters. The variable size trocar 10ⁱ, with the interchangeable guide tube 12 and securing member 16, provides a versatile opening in the patient for insertion of the clinician's instruments.

The variable size trocar 10ⁱ provides the advantage that, once a particular length of guide tube 12 is chosen, if it becomes apparent that the trocar 10ⁱ must be further inserted into the patient, the guide tube 12 can be axially (i.e., longitudinally) advanced relative to the securing member 16. It is generally desired that the guide tube 12 chosen for use by the clinician should have a length just long enough to get through the superficial fat layer and have a diameter to match the tissue. That is, larger diameters are good for holding back fat and are preferred in general when the tissue is loose and fatty, while a smaller diameter is preferred when the tissue is tight and lean.

The variable size trocar 10ⁱ meets both these needs, thus providing degrees of freedom in both tube length and tube diameter. For example, in certain embodiments, the desired lengths of the guide tubes described herein can range about 40 to about 300 mm and the diameters can range from about 10 to about 40 mm.

In the embodiment shown in FIG. 1, the securing member 16 is co-axially positioned over the guide tube 12. The securing member 16 includes an annular segment 30, a clipping member 32 and a biasing member 34.

The annular segment 30 defines an axially extending opening 31 that has a preferred internal circumference for receiving the guide tube 12, as further described below. The guide tube 12 of the trocar 10ⁱ is constructed so that it can be longitudinally moved in the opening 31 of the annular member 32.

The biasing member 34 is in contact with the clipping member 32 and provides a biasing force to the clipping member 32, as further described below.

The clipping member 32 has a front end portion 32a and a base end portion 32b that is configured to be at an angle with respect to the front end portion 32a. The biasing member 34 can be generally arranged to be at a desired fulcrum point on the base end portion 32b.

The biasing member 34 holds, or biases, the front end portion 32a of the clipping member 32 toward the outer surface of the guide tube 12. In order to position the trocar in the patient, the base end portion 32b of the clipping member 32 is pressed in a downward direction such that the front end portion 32a is moved away from the outer surface of the guide tube 12. In use, the tissue of the patient is secured between the front end portion 32a of the clipping member 32 and the outer surface of the guide tube 12.

In certain non-limiting embodiments the clipping member 32 also can include an engaging face 32c that is formed on the front end portion 32a. The engaging face 32c can have any suitable configuration that aids in securing the trocar 10 to the patient's skin.

Also, in certain non-limiting embodiments, the trocar 10ⁱ can include a sealing member 14 that is axially positioned on the proximal end 24 of the guide tube 12. There is no special limitation on the material that can be used for the sealing member 14. In certain embodiments, the sealing member 14 can comprise an elastic material such that an inner diameter of the sealing member 14 is smaller than that of the guide tube 12 and the sealing member 14 can be fit over the proximal end 24. In certain embodiments, the sealing member 14 provides an airtight state for the trocar 10. In certain embodiments, it is desired that the sealing member 14 be made of a material that has a required elasticity, air tightness, and durability. Silicone rubber, latex rubber, polyurethane, and copolymer of ethylene and vinyl acetate can be enumerated for example, and silicone rubber can meet these requirements.

Also, in certain non-limiting embodiments, the securing member 16 can further include a tube securing mechanism 33. For example, as shown in FIG. 1, the tube securing mechanism 33 includes a radially extending aperture 34 that receives a fastener such as a thumb screw 35.

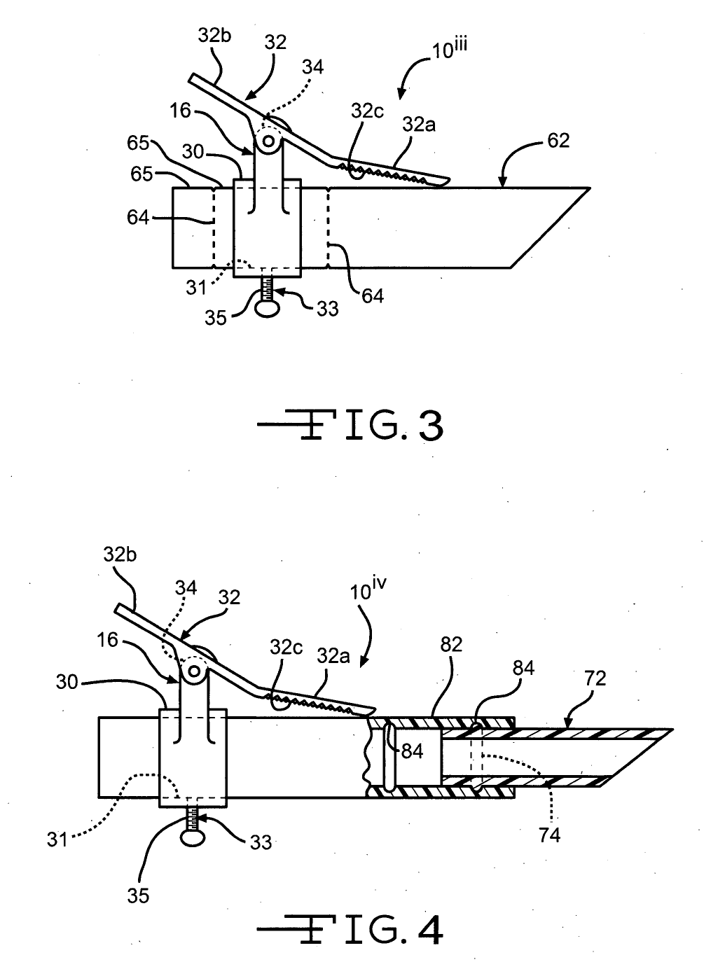

Another embodiment of a variable size trocar 10ⁱⁱ is shown in FIG. 2. It is to be noted that, for the same or similar structures as shown in FIG. 1, the same reference numbers will be used for ease of illustration. As such, in the embodiment in FIG. 2, a variable size trocar 10ⁱⁱ can further include a second guide tube 52 that is co-axially positioned within the annular member 30 of the securing member 16. The first guide tube 12 can fit snugly inside the second guide tube 52, thereby providing for a telescoping feature to the trocar 10ⁱⁱ. The embodiment shown in FIG. 2 allows the clinician to use both small and large tubes 12 and 52 together (e.g., telescoping), the large guide tube 52 only (e.g., small tube removed), or only the small guide tube 12.

In certain embodiments, when the trocar 10ⁱⁱ has the telescoping feature, the larger guide tube 52 can have a slight inner diameter tapered surface 54, greatly exaggerated in FIG. 2 for the sake of clarity, so that the first, and smaller, guide tube 12 is held by the taper of the larger guide tube 52.

Also, the second guide tube 52 can be configured to receive the tube securing mechanism 33. The second guide tube 52 can include a radially extending aperture 56 that receives the fastener 35.

Further, while not schematically illustrated in the other FIGURES herein, it is within the scope of the present invention that the other embodiments can also optionally include a tube securing mechanism to prevent the guide tube from being completely dislodged from the securing member 16 in a distal direction.

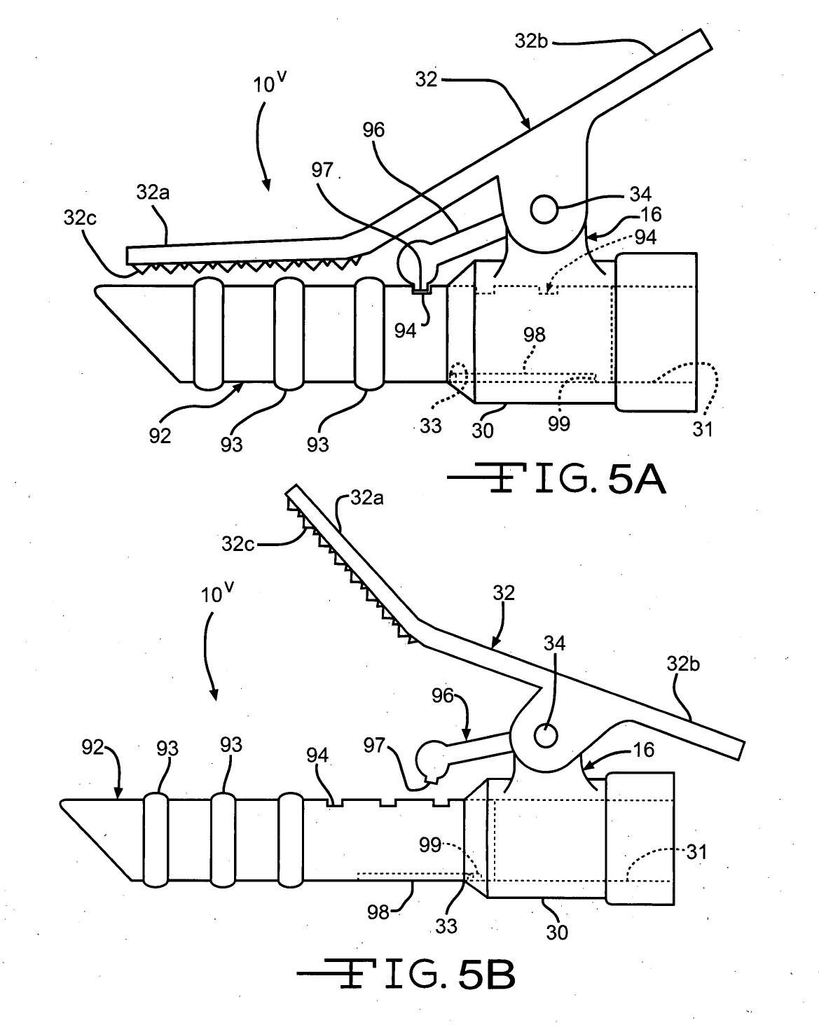

Another embodiment of a variable size trocar 10ⁱⁱⁱ is shown in FIG. 3. It is again to be noted that, for the same or similar structures as shown in FIG. 1, the same reference numbers will be used for ease of illustration. As such, in the embodiment in FIG. 3, a variable size trocar 10ⁱⁱⁱ can further include an adjustable length guide tube 62 that is co-axially positioned in the securing member 16. The adjustable length guide tube 62 includes one or more score lines 64.

The desired position of the trocar 10ⁱⁱⁱ is determined by the clinician. Once the desired position is determined, the adjustable length guide tube 62 can be longitudinally moved with respect to the securing member 16 by the clinician. The clinician can break off one or more proximal portions 65 of the adjustable length guide tube 62 at the desired score line 64 so that the trocar 10ⁱⁱ can be axially positioned to the desired depth. In such embodiments, it may be desired that no tube length protrude out of the back of the securing member 16. The lack of any protruding tube length provides more room for the surgical instruments to be fully inserted into the patient.

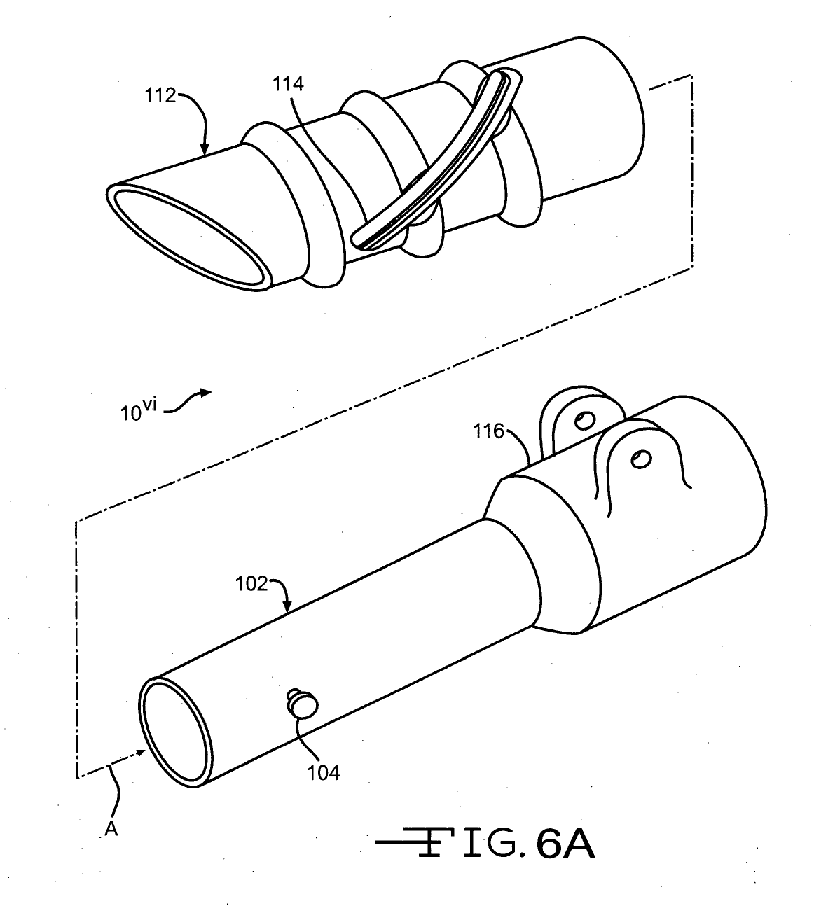

Yet another embodiment of a variable size trocar 10^{iv} is shown in FIG. 4. It is still again to be noted that, for the same or similar structures as shown in FIG. 1, the same reference numbers will be used for ease of illustration. In the embodiment in FIG. 4, a first guide tube 72 is co-axially positioned within a second guide tube 82. The first guide tube 72 can fit snugly inside the second guide tube 82, thereby providing for a telescoping feature. Additionally, the small, inner first guide tube 72 can include one or more ridges 74 that can be matingly received in a groove 84 in the inner surface of the second guide tube 82, if desired. The embodiment shown in FIG. 4 allows the clinician to use both tubes 72 and 82 and/or to move one tube with respect to the other.

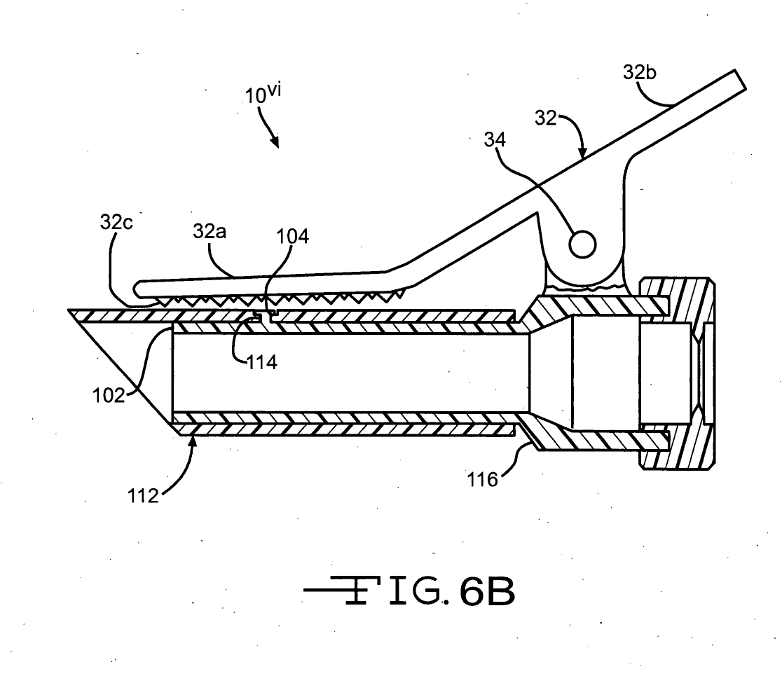

In yet another embodiment, a variable size trocar 10^{v} is shown in FIG. 5. It is again to be noted that, for the same or similar structures as shown in FIG. 1, the same reference numbers will be used for ease of illustration. As such, in the embodiment in FIG. 5, a variable size trocar 10^{v} can further include a lockable guide tube 92 that is co-axially positioned in the securing member 16. In certain embodiments, the lockable guide tube 92 can include one or more ridges 93 on its outer surface to aid in preventing the trocar 10^{v} from slipping out of the patient. In certain embodiments, the ridges 93 can have a, corrugated configuration such that the guide tube 92 can be collapsed or lengthened to a desired extension.

Further, while not schematically illustrated in the other FIGURES herein, it is within the scope of the present invention that the other embodiments can also optionally include one or more ridges to prevent the guide tube from being disengaged from the patient and/or to be extended to a desired depth within the patient.

The lockable guide tube 92 includes one or more channels 94 that at least partially extend around the circumference of the outer surface of the lockable guide tube 92. In the embodiment shown, the channel 94 has a desired externally extending cross-sectional shape that does not radially extend through the guide tube 92.

The variable size trocar 10^{v} also includes a locking member 96 that is configured to be engaged at least partially in one or more channels 94. In certain embodiments, the locking member 96 can include one or more protrusions 97 that have a cross-sectional shape that is complementary to the channel 94. In the embodiment shown, the locking member 96 is pivotably mounted to the securing member 16. It is to be understood, however, in other non-limiting embodiments, that the locking member 96 can be suitably fastened to the securing member 16 in any suitable manner.

In the embodiment shown in FIGS. 5A and 5B, the certain embodiments, the lockable guide tube 92 includes a keyway 98 and a stop 99 that prevents the lockable guide tube 92 from rotating in the axially extending opening 31 in the annular member 30 of the securing member 16. In certain embodiments, the annular member 30 can include a lip or edge 33 against which the stop 99 is engaged when the lockable guide tube 92 is in a fully extended position, as schematically illustrated in FIG. 5B. Further, while not schematically illustrated in the other FIGURES herein, it is within the scope of the present invention that the other embodiments can also optionally include a keyway and stop mechanism to prevent the guide tube from being completely dislodged from the securing member 16 in a distal direction.

In use, the desired position of the trocar 10^{v} is determined by the clinician. Once the desired position is determined, the lockable guide tube 92 can be longitudinally moved with respect to the securing member 16 by the clinician. The clinician then moves the locking member 96 in a direction toward the guide tube 92 such that at least the protrusion 97 is engaged in the channel 94. In certain embodiments, the biasing member 34 can also bias, or hold, the locking member 96 in position against the guide tube 92. In other embodiments, the complementary shapes of the channel 94 and the protrusion 97 provide an interference fit that substantially holds the guide tube 92 in a stationary position within the securing member 16.

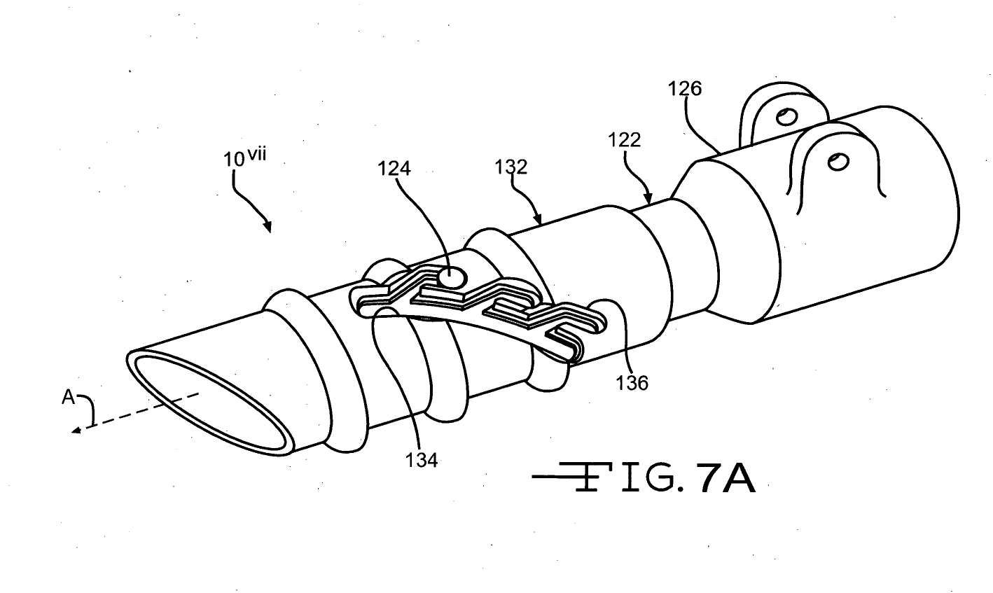

Still another embodiment of a variable size trocar 10^{vi} is shown in FIGS. 6A and 6B. It is to be noted that, for the same or similar structures as shown in FIG. 1, the same reference numbers will be used for ease of illustration. As such, in the embodiment in FIGS. 6A and 6B, a variable size trocar 10^{vi} can further include a first guide tube 102 that is co-axially positioned within a second guide tube 112. The first guide tube 102 can fit snugly inside the second guide tube 112, thereby providing for a telescoping feature. The first and second guide tubes 102 and 112 can be circumferentially rotated relative to one another along a common axis A. That is, the first and second guide tubes 102 and 112 can be twisted, or turned, relative to one another, as further described below.

The first guide tube 102 includes one or more projections 104. For ease of explanation herein, only one projection 104 is illustrated. The projection 104 extends in a generally radially outward direction from the outer surface of the first guide tube 102.

The second guide tube 112 is configured to receive the projection 104. The second guide tube 112 includes one or more apertures 114 that receive a projection 104. In one non-limiting embodiment, the aperture 114 has a generally spirally extending configuration.

The desired position of the trocar 10^{vi} is determined by the clinician. Once the desired position is determined, the second guide tube 112 can be longitudinally moved with respect to the first guide tube 102 by twisting or turning the second guide tube 112. As the clinician turns, or “unscrews”, the second guide tube 112, the projection 104 is moved along the aperture 114 in the second guide tube 112. The spiral shape of the aperture 114 allows the first and second guide tubes 102 and 112 to be twisted, or turned, relative to one another.

In certain embodiments, as shown in FIGS. 6A and 6B, the first guide tube 102 can be integrally formed with a securing member 116. Also, the securing member 116 can include the same clipping member 32 and/or biasing member 34 as shown in other FIGURES herein.

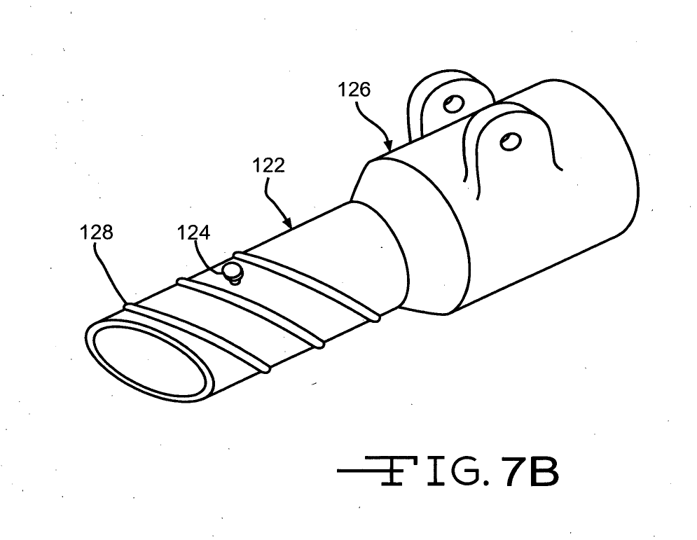

Yet another embodiment of a variable size trocar 10^{vii} is shown in FIGS. 7A and 7B. It is again to be noted that, for the same or similar structures as shown in FIG. 1, the same reference numbers will be used for ease of illustration.

As such, in the embodiment in FIGS. 7A and 7B, a variable size trocar 10^{vii} can further include a first guide tube 122 that is co-axially positioned within a second guide tube 132. The first guide tube 122 can fit snugly inside the second guide tube 132, thereby providing for a telescoping feature. The first and second guide tubes 122 and 132 can be circumferentially rotated relative to one another along a common axis A. That is, the first and second guide tubes 122 and 132 can be twisted, or turned, relative to one another, as further described below.

The first guide tube 122 includes one or more projections 124. For ease of explanation herein, only one projection 124 is illustrated. The projection 124 extends in a generally radially outward direction from the outer surface of the first guide tube 122.

The second guide tube 132 is configured to receive the projection 124. The second guide tube 132 includes one or more apertures 134 that receive a projection 124. In one non-limiting embodiment, the aperture 134 has a generally spirally extending configuration.

The aperture 134 includes one or more locking grooves 136 that extend at an angle from the aperture 134 on the outer surface of the second guide tube 132. In the embodiment shown in FIG. 7A, the locking grooves 136 have a further angled shape.

In certain embodiments, as shown in FIGS. 7A and 7B, the first guide tube 122 can be integrally formed with a securing member 126. Also, the securing member 126 can include the same clipping member 32 and/or biasing member 34 as shown in other FIGURES herein.

Further, in certain non-limiting embodiments, the first guide tube 122 can include a spirally extending thread 128 on its outer surface for engagement with an inner surface of the second guide tube 132. Further, while not schematically illustrated in the other FIGURES herein, it is within the scope of the present invention that the other embodiments can also optionally include spirally extending threads to prevent the guide tube from being completely dislodged from the second guide tube in a distal direction.

The desired position of the trocar 10^{vii} is determined by the clinician. Once the desired position is determined, the second guide tube 132 can be longitudinally moved with respect to the first guide tube 122 by twisting or turning the second guide tube 132. As the clinician turns, or “unscrews”, the second guide tube 132, the projection 124 is moved along the aperture 134 in the second guide tube 132. The spiral shape of the aperture 134 allows the first and second guide tubes 122 and 132 to be twisted, or turned, relative to one another.

Once the desired extension of the second guide tube 132 is determined by the clinician, the projection 124 is at least temporarily secured within the locking groove 136. The seating of the projection 124 in the locking groove 136 substantially prevents the now-locked second guide tube 132 from longitudinally moving with respect to the first guide tube 122.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A trocar comprising:
at least one guide tube having a desired length and outer dimension; and
at least one securing member having an annular member with an inner dimension that is complementary with the outer dimension of the guide tube;
wherein at least a portion of the guide tube is configured to be longitudinally movable with respect to the annular member of the securing member.

2. A trocar according to claim 1, wherein the securing member is configured to accept guide tubes that have one or more of different lengths and different diameters.

3. A trocar according to claim 1, wherein the securing member further includes a guide tube securing mechanism.

4. A trocar according to claim 1, wherein a second guide tube is co-axially positioned within the first guide tube.

5. A trocar according to claim 4, wherein the second guide tube has an inner diameter tapered surface, wherein the first guide tube is at least temporarily secured by the tapered surface of the second guide tube.

6. A trocar according to claim 1, wherein the guide tube comprises an adjustable length guide tube.

7. A trocar according to claim 6, wherein the adjustable length guide tube includes one or more score lines.

8. A trocar according to claim 1, including a first guide tube co-axially positioned within a second guide tube, the first guide tube including one or more ridges that are matingly received in one or more grooves on an inner surface of the second guide tube.

9. A trocar according to claim 1, wherein the guide tube comprises a lockable guide tube, the lockable guide tube including one or more externally extending channels, and wherein the trocar further including a locking member that is configured to be at least partially engaged in one or more channels in the lockable guide tube.

10. A trocar according to claim 9, wherein the locking member is configured to be movably fastened to the securing member.

11. A trocar according to claim 1, including a first guide tube and a second guide tube that is co-axially positioned within the first guide tube, wherein at least one of the first and second guide tubes are configured to be circumferentially rotated relative to one another.

12. A trocar according to claim 11, wherein the first guide tube includes one or more projections and the second guide tube is configured to receive the one or more projections.

13. A trocar according to claim 12, wherein the projection extends in a generally radially outward direction from an outer surface of the first guide tube.

14. A trocar according to claim 12, wherein the second guide tube includes at least one aperture that receives one or more projections.

15. A trocar according to claim 14, wherein the aperture has a generally spirally extending configuration.

16. A trocar according to claim 1, including a first guide tube and a second guide tube that is co-axially positioned around the first guide tube;
the first guide tube having one or more projections extending in a generally radially outward direction from an outer surface of the first guide tube;
the second guide tube having one or more apertures that are configured to receive the one or more projections; the aperture having one or more locking grooves that extend at an angle from the aperture on the outer surface of the second guide tube.

17. A trocar according to claim 16, wherein the locking groove is positioned at an angle with respect to the aperture.

18. A trocar according to claim 16, wherein at least one projection is configured to be at least temporarily secured within the locking groove and is configured to prevent the second guide tube from longitudinally moving with respect to the first guide tube.

19. A trocar according to claim 1, wherein the guide tube includes a mechanism configured to prevent the guide tube from being completely dislodged from the securing member in a distal direction.

20. A trocar according to claim 1, wherein the guide tube has a corrugated configuration.
